(19) United States
(12) Patent Application Publication
(10) Pub. No.: US 2025/0334515 A1
(43) Pub. Date: Oct. 30, 2025

(54) BIOFILM THICKNESS MEASURING DEVICE

(71) Applicant: THE WAVE TALK, INC., Daejeon (KR)

(72) Inventors: Young Dug KIM, Seongnam-si Gyeonggi-do (KR); Kyoung Man CHO, Seoul (KR)

(73) Assignee: THE WAVE TALK, INC., Daejeon (KR)

(21) Appl. No.: 19/188,378

(22) Filed: Apr. 24, 2025

(30) Foreign Application Priority Data
Apr. 25, 2024 (KR) 10-2024-0055560
Apr. 24, 2025 (KR) 10-2025-0053762

(51) Int. Cl. G01N 21/47; C12Q 1/04; G01N 21/41; G01N 21/94

(52) U.S. Cl. CPC G01N 21/4788; C12Q 1/04; G01N 21/41; G01N 2021/479; G01N 2021/945

(57) ABSTRACT

Provided is a biofilm thickness measuring device including a fluid receptacle configured to contain a target fluid, a light source configured to emit interfering input light toward the fluid receptacle, a first detector configured to detect a speckle of output light produced by multiple scattering of the emitted input light in the target fluid, a second detector configured to detect an intensity of the output light, and a controller configured to calculate thickness information of a biofilm in the fluid receptacle using the detected speckle and the detected intensity of the output light.

### CROSS-REFERENCES TO RELATED APPLICATIONS

This application claims priority to Korean Patent Application No. 10-2024-0055560 filed on Apr. 25, 2024 and Korean Patent Application No. 10-2025-0053762 filed on Apr. 24, 2025, the entire contents of which are herein incorporated by reference.

### TECHNICAL FIELD

Embodiments of the disclosure related to a biofilm thickness measuring device.

### DESCRIPTION OF THE RELATED ART

Turbidity measuring devices are widely used in a variety of applications such as water supply systems, sewer systems, industrial water systems, and water treatment facilities, to manage water quality in real time. Turbidity is an indicator of the concentration of particulate matter suspended in water and is measured using optical sensors based on the scattering and absorption properties of particles.

However, conventional turbidity measurement methods using optical sensors may only measure macroscopic turbidity and are limited by the inability to accurately detect impurities or minute amounts of microorganisms in water.

To overcome these technical limitations, a speckle detection method has recently been developed and used to detect contamination by bacteria or microorganisms cultured in a medium in a non-contact manner by capturing a speckle pattern of the medium caused by multiple scattering by bacteria or microorganisms in the medium by a camera using laser light generated by a laser source.

However, in pipes or the like where actual turbidity measurement is required, biofilms caused by the adhesion and proliferation of microorganisms are often encountered and reduce the accuracy of speckle detection. Biofilms consist of microbial communities, such as bacteria, algae, and fungi, and a mucilaginous substrate produced by the biofilms, and gradually thicken over time. In case that biofilms coat the inside of a pipe, biofilms interfere with the transmission or scattering of light, thereby reducing the accuracy and reliability of turbidity measurements.

Conventionally, periodic cleaning or replacement of turbidity measurement devices has been used to reduce the impact of such biofilms, but these methods are inefficient, are costly to maintain, and have limitations for continuous condition detection. In particular, the lack of technology for quantitatively measuring or detecting the thickness of biofilms makes real-time response and maintenance difficult.

Therefore, there is a need for technology which may quantitatively and continuously monitor the condition of biofilms forming in the inside of a pipe, and in particular, there is a growing need for devices which may accurately measure the thickness of biofilms.

### SUMMARY

### Problems to be Solved

Embodiments of the disclosure are intended to address the problems and/or limitations described above, and aim to provide a biofilm thickness measuring device able to obtain information about the condition of biofilms adhering to the inner wall of the measuring device.

However, these objectives are exemplary and do not limit the scope of the disclosure.

### Means to Solve the Problems

An embodiment of the disclosure provides a biofilm thickness measuring device including: a fluid receptacle configured to contain a target fluid; a light source configured to emit interfering input light toward the fluid receptacle; a first detector configured to detect a speckle of output light produced by multiple scattering of the emitted input light in the target fluid; a second detector configured to detect an intensity of the output light; and a controller configured to calculate thickness information of a biofilm in the fluid receptacle using the detected speckle and the detected intensity of the output light.

In an embodiment of the disclosure, the controller may further be configured to obtain the intensity and the speckle of the output light in a time series sequence, and calculate refractive index information of the biofilm using a temporal change in the intensity of the output light and a temporal change in the speckle of the output light.

In an embodiment of the disclosure, the controller may further be configured to calculate the refractive index information by comparing the intensity of the output light measured at a first time point before formation of the biofilm on an inner surface of the fluid receptacle and the intensity of the output light measured at a second time point after the formation of the biofilm on the inner surface of the fluid receptacle.

In an embodiment of the disclosure, the controller may further be configured to calculate a thickness of the biofilm using the calculated refractive index information of the biofilm.

In an embodiment of the disclosure, the controller may further be configured to obtain a temporal correlation of the speckle using the detected speckle of the output light, and calculate a concentration of a target material in the target fluid based on the obtained temporal correlation.

In an embodiment of the disclosure, the temporal correlation may include a difference between first image information of the speckle detected at a first time point and second image information of the speckle detected at a second time point different from the first time point.

In an embodiment of the disclosure, the first image information and the second image information may include pattern information of the speckle.

In an embodiment of the disclosure, the controller may further be configured to obtain a spatial correlation of the speckle using the detected speckle of the output light, and calculate a concentration of a target material in the target fluid based on a temporal change of the obtained spatial correlation.

Another embodiment of the disclosure provides a biofilm thickness measuring device including: a fluid receptacle configured to contain a target fluid; a light source configured to emit interfering input light toward the fluid receptacle; a detector configured to obtain an image of output light produced by multiple scattering of the emitted input light in the target fluid; and a controller configured to obtain a speckle and an intensity of the output light using the obtained image of the output light, and calculate thickness information of a biofilm in the fluid receptacle using the obtained speckle and the obtained intensity of the output light.

In another embodiment of the disclosure, the detector may obtain a plurality of images by measuring the output light in a time series sequence, and the controller may further be configured to obtain an intensity of the output light by comparing the image measured at a first time point before formation of the biofilm on an inner surface of the fluid receptacle and the image measured at a second time point after the formation of the biofilm on the inner surface of the fluid receptacle among the plurality of images.

Aspects, features, and advantages other than the foregoing will become apparent from the following drawings, claims, and detailed description of the disclosure.

### Technical Effects of the Invention

According to the above-described means for achieving the objectives of the disclosure, embodiments of the disclosure may accurately calculate thickness information of a biofilm generated in a measuring device in real time.

Embodiments of the disclosure may predict in advance a time at which maintenance of the turbidity measuring device is to be performed using the thickness information of the biofilm, thereby improving the maintenance efficiency of the device and reducing management costs.

The scope of the disclosure is not limited to these effects.

### BRIEF DESCRIPTION OF THE DRAWINGS

These and/or other aspects will become apparent and more readily appreciated from the following description of the embodiments, taken in conjunction with the accompanying drawings in which:

FIG. 1 is a side cross-sectional view conceptually illustrating a biofilm thickness measuring device according to an embodiment of the disclosure;

FIG. 2 is a view illustrating a state in which a biofilm is generated on the biofilm thickness measuring device according to an embodiment of the disclosure;

FIG. 3 is a view illustrating a measuring principle of the biofilm thickness measuring device according to an embodiment of the disclosure;

FIG. 4 is a flowchart sequentially illustrating a biofilm thickness measuring method according to an embodiment of the disclosure;

FIGS. 5 and 6 are conceptual views schematically illustrating a biofilm thickness measuring method according to another embodiment of the disclosure; and

FIGS. 7 and 8 are conceptual views schematically illustrating a biofilm thickness measuring device according to another embodiment of the disclosure.

### DETAILED DESCRIPTION

Hereinafter, the following embodiments are described in detail with reference to the accompanying drawings, and in the description with reference to the drawings, the same reference numerals are given to the same or corresponding elements, and repeated description thereof is omitted.

The embodiments may have various modifications, and thus specific embodiments will be shown in the drawings and described in detail in the detailed description. The effects and features of the embodiments and how to accomplish the same will be apparent with reference to the following detailed description together with the drawings. However, the embodiments are not limited to the embodiments disclosed below, but may be implemented in various forms.

To clearly describe the disclosure, parts not relevant to the description are omitted from the drawings, and the same reference numerals may be used for similar elements throughout the specification.

In the following embodiments, the terms such as “first” and “second” are not intended to be limiting, but are used to distinguish one element from another.

In the following embodiments, the singular forms are intended to include the plural forms as well, unless the context clearly indicates otherwise.

In the following embodiments, the term such as “comprise”, “include”, or “have” specify the presence of features or elements stated in the specification but do not preclude the possibility of addition of one or more other features or elements.

In the following embodiments, when a portion, such as a unit, a region, or a component, is referred to as being above or on another portion, the portion may be directly above or on the other portion or an intervening portion, such as a unit, a region, or a component, may also be present between the two portions.

In the following embodiments, terms, such as “connect” or “couple”, do not necessarily mean a direct and/or fixed connection or coupling of two members, unless the context clearly indicates otherwise, and do not exclude the presence of other members provided between the two members.

In the drawings, components may be exaggerated or reduced in size for ease of explanation. For example, the sizes and thicknesses of the respective components shown in the drawings are arbitrary for ease of explanation, and therefore the following embodiments are not necessarily limited to those shown.

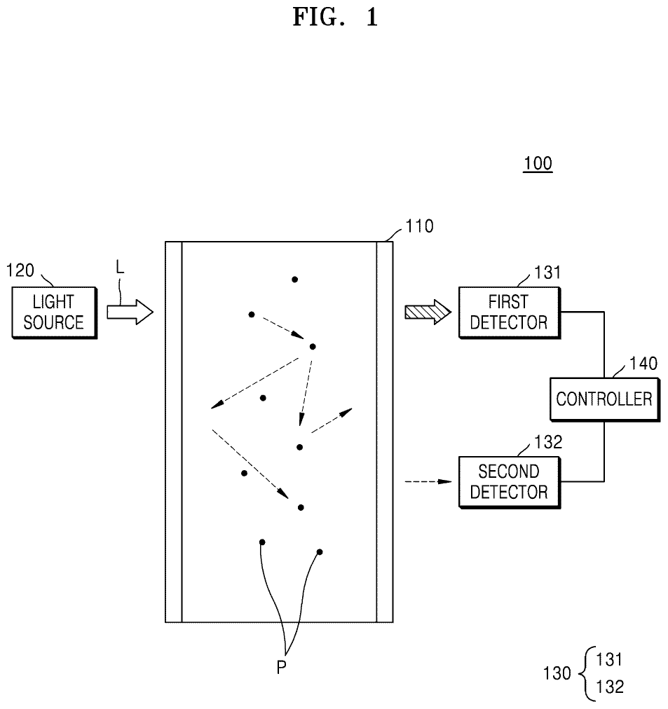

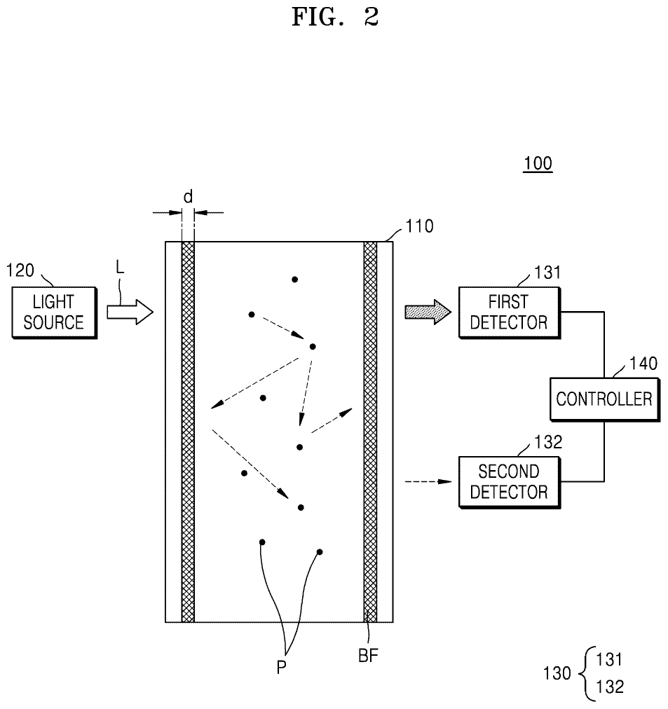

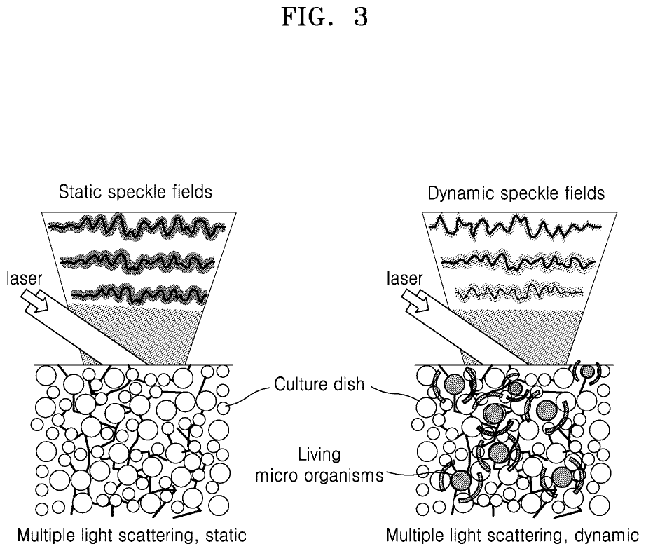

FIG. 1 is a side cross-sectional view conceptually illustrating a biofilm thickness measuring device 100 according to an embodiment of the disclosure, FIG. 2 is a view illustrating a state in which a biofilm BF is generated on the biofilm thickness measuring device according to an embodiment of the disclosure, and FIG. 3 is a view illustrating a measuring principle of the biofilm thickness measuring device 100 according to an embodiment of the disclosure.

Referring now to FIGS. 1 to 3, the biofilm thickness measuring device 100 according to an embodiment of the disclosure may include a fluid receptacle 110, a light source 120, a detector 130, and a controller 140.

The biofilm thickness measuring device 100 is a device which detects information about the biofilm BF attached to the fluid receptacle 110 using light, for example, the biofilm thickness measuring device 100 may quantitatively calculate the thickness of the biofilm BF by time series detection of the intensity and speckles of light scattered by a target fluid and the biofilm BF.

As used herein, the target fluid may be liquid or gas and may include any material in which microorganisms may grow. For example, the target fluid may be water, such as tap water or wastewater. The target fluid may contain a target material P that is a foreign matter. For example, the target material P may be a suspended solid which has a particle diameter of 2 micrometers or more and is insoluble in water or a turbid material having a particle diameter less than 2 micrometers.

The fluid receptacle 110 may contain the target fluid, and may have a light scattering space formed therein in which input light L may be multiply reflected or multiply scattered on a plurality of paths. For example, the fluid receptacle 110 may be tubular, cylindrical, or polygonal. In a case in which the fluid receptacle 110 is tubular, the target fluid may enter the fluid receptacle 110 and exit to the outside through the interior space.

In an embodiment, the fluid receptacle 110 may include at least a portion of a water system or a sewer system. The fluid receptacle 110 may be disposed at one or more locations in the water system or the sewer system to monitor a biofilm, water quality, turbidity, and the like.

In an embodiment, scattering protrusions or a scattering layer may be provided on the inner surface to allow sufficient multiple scattering to occur in the fluid receptacle 110. The scattering layer may include a scattering material, for example, the scattering layer may include hexagonal boron nitride (h-BN).

The fluid receptacle 110 may have a light entrance on a side through which the input light L is input and a light exit on an opposite side for measuring a speckle pattern generated in the scattering space.

The light source 120 may emit interfering input light L toward the fluid receptacle 110. The light source 120 may be implemented as any type of source device capable of generating light. For example, the light source 120 may be a laser capable of emitting light in a particular wavelength band. Although the light source 120 of the disclosure is not limited to a particular type, the following discussion will focus on a case in which the light source 120 is a laser for ease of explanation.

The light source 120 may use a laser having good interference to form speckles as an interference pattern in the target fluid contained in the fluid receptacle 110. In this case, the narrower the spectral bandwidth of the input light L emitted by the laser, the more accurate the measurement of the detector 130 may be. For example, the longer the coherence length of the input light L, the more accurate the measurement may be. Accordingly, a laser having a spectral bandwidth narrower than a selected reference bandwidth may be used as the light source 120, and the measurement accuracy may be higher as the spectral bandwidth is narrower than the reference bandwidth.

In an embodiment, the light source 120 may be set to maintain a condition in which the spectral bandwidth of the input light L is narrower than 5 nm. In this case, the spectral bandwidth of the input light L that irradiates the fluid receptacle 110 to measure the thickness of the biofilm BF may be maintained narrower than 5 nm.

Furthermore, the light source 120 may emit the input light L having a wavelength range that may minimize absorption in the target fluid. In an embodiment, the light source 120 may emit the input light L having a wavelength range such that the absorption coefficient of the fluid is below a selected value. For example, the input light L may have a wavelength range of 200 nm to 1.8 mm such that the absorption coefficient of water is less than 1×10³ to 1×10⁴.

The detector 130 may be disposed on the path of light emitted from the fluid receptacle 110 to detect output light. The detector 130 may detect the output light emitted from the fluid receptacle 110 and transmit the output light to the controller 140. In an embodiment, the detector 130 may include a first detector 131 and a second detector 132.

The first detector 131 may detect speckles in the output light emitted from the fluid receptacle 110. The first detector 131 may detect the output light that has passed through the fluid receptacle 110 and measure the output light as an optical image. For example, the first detector 131 may be, but is not limited to, a CCD camera, and may be implemented as various types of image sensors capable of detecting a speckle image of the output light.

Specifically, the first detector 131 uses a non-contact speckle sensing method, in which the principle of a chaotic wave sensor may be used. Describing the principle of the chaotic wave sensor with reference to FIG. 3, in case that a material having a homogeneous internal refractive index, such as glass, is irradiated with interfering light, refraction occurs in a selected direction. However, in case that an object having inhomogeneous internal refractive indices or including microscopic refracting or scattering protrusions is irradiated with coherent light such as a laser, highly complex multiple scattering occurs in the material.

As shown in FIG. 3, a portion of the light or waves (hereinafter referred to as waves for simplicity) emitted by the light source, which is scattered on a complex path by multiple scattering, passes through a target surface to be inspected. Waves passing through different points on the target surface to be inspected causes either constructive interference or destructive interference with each other, and the constructive interference or destructive interference of these waves produces a grainy pattern (i.e., speckles).

Waves scattered on these complex paths are referred to herein as “chaotic waves,” and such chaotic waves may be detected by means of interferometric speckles, which may be detected as laser speckles in a case in which the interfering light is a laser.

Again, the left part of FIG. 3 is a view showing a case in which a stable medium is irradiated with a laser beam. In case that a stable medium without movement of internal components is irradiated with interference light (e.g., a laser beam), a stable speckle pattern without change may be observed. However, as shown in the right part of FIG. 3, in a case in which the internal components include an unstable moving medium such as bacteria, the speckle pattern changes.

For example, the light path may change microscopically over time due to microscopic life activity of microorganisms (e.g., intracellular movement or movement of microorganisms). Because the speckle pattern is a phenomenon caused by interference of waves, microscopic changes in the light path may cause changes in the speckle pattern. Accordingly, by measuring the temporal changes in the speckle pattern, the movement of an organism may be quickly measured. In this manner, measuring the temporal changes in the speckle pattern may reveal the presence or absence and the concentration of microorganisms, and also the type of microorganisms.

A configuration which measures changes in such a speckle pattern is defined herein as a chaotic wave sensor.

A fluid such as water does not contain a homogeneous scattering material therein, and thus the complex multiple scattering described above does not occur in the absence of microorganisms. However, even in a fluid such as water, in case that the fluid is irradiated with waves, microscopic scattering may occur in the fluid caused by molecules or atoms in the fluid.

The degree of multiple scattering of waves in the fluid may be less than in a case in which scattering occurs in a homogeneous material, but such scattering may cause waves to interfere with each other, either constructively or destructively, and speckles may be detected by the first detector 131. In the absence of foreign matter in the target fluid, the degree to which waves are scattered by atoms or molecules in the target fluid may be constant on average in the absence of external stimulus. Here, external stimulus may refer to, for example, bubbles created in the target fluid by vibration or impact.

As shown in FIG. 2, the thickness d of the biofilm BF formed on the inner wall of the fluid receptacle 110 may increase over time. The biofilm BF is a membrane-like microbial community, which may be generated by the action of microorganisms contained in the target fluid. The biofilm BF may adhere to the inner surface of the fluid receptacle 110, grow, and increase in thickness d over time.

The biofilm thickness measuring device 100 according to an embodiment of the disclosure is configured to accurately measure the thickness d of the biofilm BF based on the relationship between the thickness d of the biofilm BF, which changes over time, and the intensity of output light and speckles.

The first detector 131 may detect time-varying speckles at preset times and provide the detected speckles to the controller 140. The first detector 131 may detect the speckles at a rate sufficient to detect microbial movement, such as at a rate of 25 frames to 30 frames per second.

The first detector 131 may measure output light in a time series sequence to obtain a plurality of images. In an embodiment, the first detector 131 may measure speckles of output light at preset times. As used herein, a time may refer to any instant in a continuous flow of time, and times may be preset at, but not necessarily limited to, equal time intervals, and may be preset at arbitrary time intervals.

For example, the first detector 131 may detect speckles of output light at a first time point before the formation of the biofilm BF in the fluid receptacle 110, detect speckles of output light at a second time point that has elapsed by a selected time from the first time point, and provide the detected speckles of output light to the controller 140. FIG. 1 may illustrate the biofilm thickness measuring device 100 at the first time point, and FIG. 2 may illustrate the biofilm thickness measuring device 100 at the second time point. The first and second time points are merely examples selected for ease of explanation, and the first detector 131 may also detect speckles of output light at the first time point, which is a reference time, and at one or more times after the first time point.

The first detector 131 may be configured for high-speed measurement in the case of measuring speckles from a flowing fluid. As used herein, high-speed measurement means detecting speckles faster than the flow velocity of the fluid. For example, the measurement speed of the first detector 131 may be set to be faster than the flow velocity at which the target fluid flows in the fluid receptacle 110.

In a case in which the first detector 131 is implemented as an image sensor, the image sensor may be arranged such that the size dd of each pixel of the image sensor is less than or equal to the grain size of the speckle pattern. In this case, a pixel size that is too small may cause undersampling, which would make it difficult for the image sensor to utilize the pixel resolution. Accordingly, to achieve an effective signal to noise ratio (SNR), the image sensor may be arranged such that the first detector 131 has at most five or less pixels located in the speckle grain size.

In an embodiment, the first detector 131 may be disposed at a different height from the height of the light source 120. For example, the first detector 131 may be disposed offset from the major axis of emission of the light source 120 which generates the input light L. Accordingly, in case that the input light L from the light source 120 is input into the fluid receptacle 110, multiple reflection and multiple scattering may be sufficiently generated in the fluid receptacle 110 to amplify changes over time caused by microscopic movement of foreign matter contained in the target fluid or bacteria or microorganisms into an overall speckle pattern, which may then be measured by the first detector 131.

The second detector 132 may measure the intensity of output light emitted from the fluid receptacle 110. For example, the second detector 132 may be a photodiode. The second detector 132 may measure the intensity of output light in a time series sequence to obtain the time-varying intensity of the output light.

In an embodiment, the second detector 132 may measure the intensity of output light at the same time as the first detector 131 and transmit the measured intensity of output light to the controller 140. For example, the second detector 132 may measure the intensity of output light at the first time point before the formation of the biofilm BF in the fluid receptacle 110, may measure the intensity of output light at the second time point that has elapsed by a selected time from the first time point, and provide the measured intensities to the controller 140. However, the disclosure is not limited thereto, and the second detector 132 may measure the intensity of output light at a different time from the measurement time of the first detector 131.

In case that the fluid receptacle 110 is irradiated with the input light L, the input light L is multiply scattered by the interaction of the input light L with the target fluid, and the second detector 132 may measure the intensity of output light, i.e., scattered light, emitted from the fluid receptacle 110. As the concentration of the target material P in the target fluid increases, the scattering of light at a selected angle or in a selected direction increases, and the intensity of the output light emitted toward the second detector 132 may increase accordingly.

In a case in which the amount of the target material P in the target fluid is constant without changes in the biofilm BF, the intensity of output light measured by the second detector 132 may be constant over time. In comparison, in a case in which the biofilm BF is generated on the inner surface of the fluid receptacle 110, the intensity of output light measured by the second detector 132 may change over time even in a case in which the amount of the target material P in the target fluid is constant.

Specifically, in a case in which the biofilm BF is created on the inner surface of the fluid receptacle 110, the input light L irradiating the fluid receptacle 110 is scattered or refracted by the biofilm BF as well as the target fluid. As the thickness d of the biofilm BF attached to the inner surface of the fluid receptacle 110 increases, the scattered light increases further, and the intensity of output light emitted toward the second detector 132 increases accordingly. The second detector 132 may measure changes in the intensity of the output light over time and transmit the changes in the intensity of the output light to the controller 140.

The controller 140 may obtain output light information from the first detector 131 and the second detector 132, and may calculate the thickness of the biofilm BF in real-time using the obtained output light information. As used herein, the term “real-time” may mean estimating the thickness of the biofilm BF in 3 seconds or less, and desirably 1 second or less.

The controller 140 may obtain speckles of output light from the first detector 131 and the intensity of output light from the second detector 132. In an embodiment, the controller 140 may calculate concentration information of the target material using speckle information of the output light obtained from the first detector 131, and may estimate the thickness of the biofilm BF using the calculated concentration information of the target material and the intensity of the output light obtained from the second detector 132.

Specifically, the controller 140 may calculate the temporal correlation of speckles using the difference between first image information of speckles detected by the first detector 131 at the first time point and second image information of speckles detected at the second time point, and may estimate the concentration of the target material P using the calculated temporal correlation. Here, the first time point may be a time before the formation of the biofilm BF on the inner surface of the fluid receptacle 110, and the second time point may be a time after the formation of the biofilm BF on the inner surface of the fluid receptacle 110.

The first image information and the second image information may include pattern information of speckles. An embodiment of the disclosure is not limited to using the difference between the first image information at the first time point and the second image information at the second time point, but may be extended to using the image information of a plurality of speckles at a plurality of times.

The controller 140 may calculate the temporal correlation coefficient of images using the image information of speckles generated at a plurality of preset time points, and may estimate the concentration of the target material P in the target fluid based on the temporal correlation coefficient. The temporal correlation of detected speckles may be calculated using Equation 1 below. However, Equation 1 below is only an example, and other equations may be used to derive the temporal correlation of speckles.

$$\overline{C} ( x , y ; τ ) = \frac{1}{T - τ} {\sum }_{t = 1}^{T - τ} \overline{I} ( x , y ; t ) \overline{I} ( x , y ; t + τ ) δ t \qquad Equation ( 1 )$$

In Equation 1, Image indicates a temporal correlation coefficient, Image indicates a normalized light intensity, (x, y) indicates pixel coordinates of a camera, t indicates a measured time, T indicates a total measurement time, and T indicates a time lag.

The temporal correlation coefficient may be calculated according to Equation 1, and in an embodiment, the concentration of the target material P in the target fluid may be estimated by an analysis in which the temporal correlation coefficient is a preset threshold value or less. Furthermore, the controller 140 may estimate the concentration of the target material P in the target fluid using the rate of change or the peak value of the temporal correlation coefficient.

In another embodiment, the controller 140 may obtain a spatial correlation of speckles. Here, the spatial correlation given by the following Equation may be represented as a constant range of numbers indicating the similarity of the brightness of an arbitrary pixel and the brightness of another pixel at a distance r from the arbitrary pixel in an image, measured at a time t. The range may be a range of −1 to 1. For example, the spatial correlation indicates the degree of correlation between the arbitrary pixel and the other pixel, where 1 indicates a positive correlation, −1 indicates a negative correlation, and 0 indicates no correlation. Specifically, before the interference pattern is formed, the brightness of emission is uniform and thus the spatial correlation of a sample image shows a positive correlation close to 1, but after the interference pattern is formed, the correlation value may drop to a value close to 0.

A brightness measured in a pixel at a position r′=(x,y) at a time t by the first detector 131 may be defined as l(r′,t), and a brightness of another pixel at a distance r from the pixel at the position r′=(x,y) may be defined as l(r′+r,t). A spatial correlation defined using the brightnesses may be represented by Equation 2.

$$C ( r , t ) = \frac{1}{{C}_{0} ( t )} \int \int I ( {r}^{′} + r , t ) I ( {r}^{′} , t ) {dr}^{′} \qquad Equation ( 2 )$$

C₀(t) was used to fit Equation 4 to the range of −1 to 1. In case that the brightness l(r′,t) measured in the arbitrary pixel at the time t is equal to the brightness l(r′+r,t) of the other pixel at the distance r from the arbitrary pixel, 1 is derived as the spatial correlation, otherwise a value less than 1 is derived.

In an embodiment, the disclosure may represent the spatial correlation as a function of time only. In this regard, the controller 140 may average the spatial correlation over pixels having the same magnitude r from the arbitrary pixel, as shown in Equation 3 below.

$$C ( ρ , t ) = \frac{1}{2 π} {\int }_{0}^{2 π} C ( r , t ) d θ \qquad Equation ( 3 )$$

In an embodiment, the controller 140 may represent the preset distance as a function of time by substituting the preset distance into Equation 3 above, and may use the function to determine the degree to which the interference pattern is formed as a value in the range of 0 to 1.

The controller 140 may determine the concentration information of the target material P using the spatial correlation as follows. The spatial correlation may be obtained by generating two identical superimposed images from a single image, shifting one of the two images by a preset distance in a single direction, and analyzing the similarity of two adjacent pixels between the shifted image and the non-shifted image. Here, the spatial correlation is a measure of the uniformity of images, and in case that interference patterns are formed due to a suspended or turbid material, the similarity of two adjacent pixels may be reduced by small interference patterns, thereby reducing the value of the spatial correlation.

The spatial correlation coefficient is dependent on the shifted distance r, in which in a selected distance range, the value decreases as the shifting distance r increases, and beyond the selected distance range, the value is approximately constant. Therefore, to obtain a more meaningful spatial correlation, the controller 140 may obtain the spatial correlation by shifting the image beyond the distance. In this case, the selected distance r is dependent on the speckle size, and in case that an image is represented on a pixel-by-pixel basis, the controller may obtain the spatial correlation by shifting the image by a pixel that is larger than the speckle size.

The controller 140 may calculate information about the thickness d of the biofilm BF using the calculated concentration information of the target material P and the intensity of output light obtained by the second detector 132.

The change of the speckle image caused by the biofilm BF is significantly slower than the change of the speckle image caused by the target material P contained in the target fluid. Therefore, for the temporal correlation calculated from the difference between the speckle images obtained at the first and second time points, only the speckle information caused by the target material P may be considered, and the speckle information caused by the biofilm BF may be ignored.

In comparison, the intensities of the output light obtained by the second detector 132 at the first time point and the second time point include both the intensity information of scattered light caused by the target material P and the intensity information of scattered light caused by the biofilm BF. Therefore, information about the biofilm BF may be obtained using both the speckle of the output light obtained by the first detector 131 and the intensity of the output light obtained by the second detector 132.

The controller 140 may calculate the thickness of the biofilm BF using the calculated concentration information of the target material P and the intensity information of the output light obtained by the second detector 132.

Light traveling through the receptacle 110 is refracted by the biofilm BF, and as the thickness d of the biofilm BF increases, the refractive index of the biofilm BF changes. The controller 140 may calculate the intensity information of the output light that changes over time using the intensity of the output light obtained by the second detector 132, and calculate refractive index information of the biofilm BF using the calculated intensity information of the output light.

The controller 140 may calculate the thickness d of the biofilm BF using the calculated refractive index information of the biofilm BF and the correlation between the thickness d and the refractive index of the biofilm BF.

The controller 140 may also derive the material type of the biofilm BF using the refractive index of the biofilm BF at a particular time from the calculated refractive index information of the biofilm BF.

The controller 140 may provide the information about the biofilm BF in real time, including at least one of the presence or absence of the biofilm BF, the thickness d of the biofilm BF, or the material type of the biofilm BF.

Furthermore, the controller 140 may monitor whether the biofilm thickness measuring device 100 is operating normally using the estimated information about the thickness d of the biofilm BF. The controller 140 may also determine a time at which maintenance of the biofilm thickness measuring device 100 is desired and generate an alarm or a report related to the maintenance using the information about the thickness d of the biofilm BF.

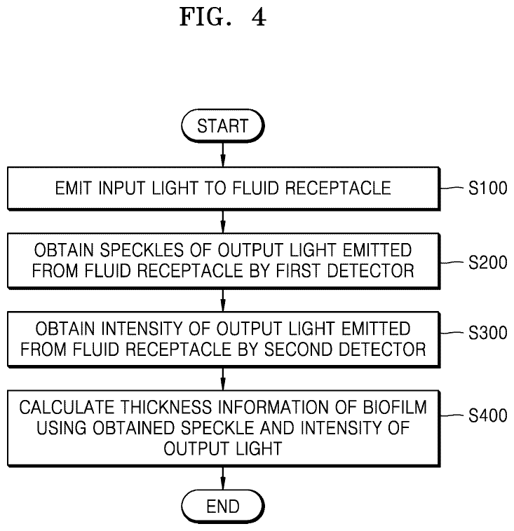

FIG. 4 is a flowchart illustrating a biofilm thickness measuring method according to an embodiment of the disclosure.

Referring to FIG. 4, the biofilm thickness measuring method according to an embodiment of the disclosure may include emitting input light to a fluid receptacle (S100), obtaining speckles of output light emitted from the fluid receptacle by a first detector (S200), obtaining the intensity of the output light emitted from the fluid receptacle by a second detector (S300), and calculating thickness information of a biofilm using the obtained speckle and the obtained intensity of the output light (S400).

First, the input light is emitted to the fluid receptacle using a light source (S100). The fluid receptacle may be provided with a scattering space in which the input light may be multiply scattered. The scattering space may contain a target fluid. The light source may be, but is not limited to, a laser having good coherence.

Thereafter, speckles of the output light emitted from the fluid receptacle are obtained by the first detector (S200). The first detector may be disposed on a path of light emitted from the fluid receptacle to detect the speckles of the output light, and may transmit the detected speckles of the output light to the controller. For example, the first detector may be a CCD camera which may detect the output light and measure the output light as an optical image.

Thereafter, the intensity of the output light emitted from the fluid receptacle is obtained by the second detector (S300). The second detector may be disposed on the path of light emitted from the fluid receptacle to measure the intensity of the output light, and may transmit the measured intensity of the output light to the controller. For example, the second detector may be a photodiode.

Thereafter, the thickness of the biofilm is calculated using the obtained speckle and the obtained intensity of the output light (S400). The controller may calculate a temporal correlation of the speckles using the speckles of the output light, and may calculate a concentration of the target material using the calculated temporal correlation. Furthermore, the controller may calculate the thickness of the biofilm using both the obtained speckles of the output light and the obtained intensity of the output light.

Because the operation principle and the operations of the biofilm thickness measuring method according to an embodiment of the disclosure are similar to those of the above-described biofilm thickness measuring device 100 and may be readily implemented and performed by a person of ordinary skill in the art, a more detailed description thereof will be omitted and reference may be made to a series of descriptions of the above-described biofilm thickness measuring device 100.

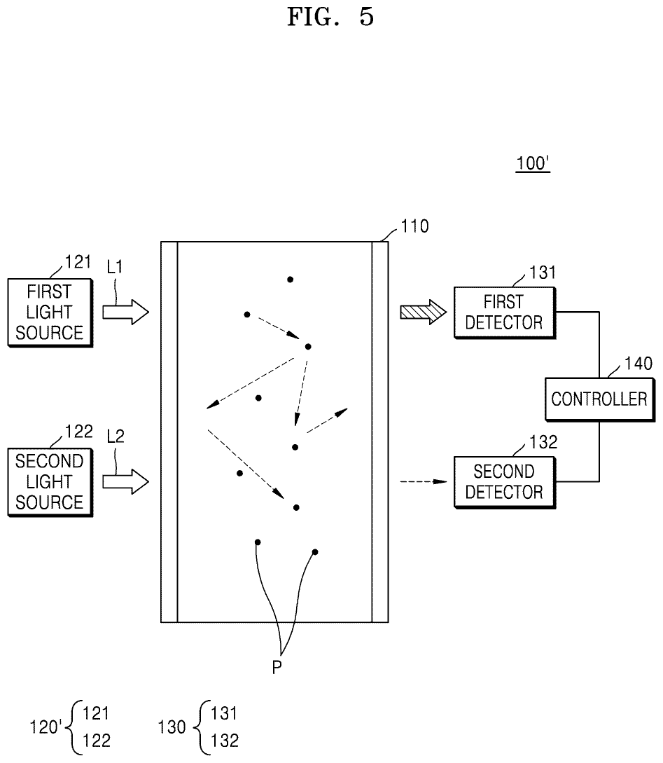

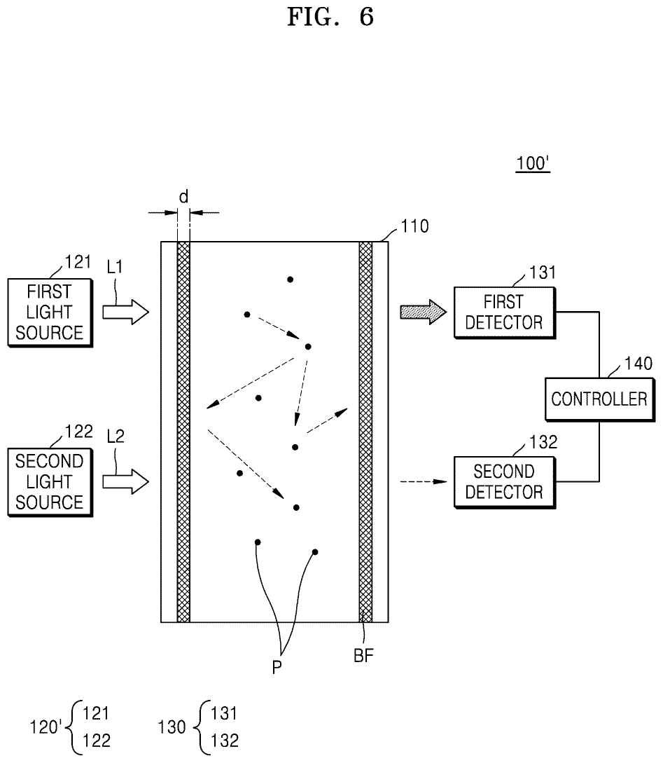

FIG. 5 and FIG. 6 are conceptual views schematically illustrating a biofilm thickness measuring device 100′ according to another embodiment of the disclosure.

Referring to FIGS. 5 and 6, the biofilm thickness measuring device 100′ according to another embodiment of the disclosure may include a fluid receptacle 110, a light source 120′, a detector 130, and a controller 140. Compared to the biofilm thickness measuring device 100 according to an embodiment described with reference to FIGS. 1 to 3, the biofilm thickness measuring device 100′ according to another embodiment shown in FIGS. 5 and 6 differs in the configuration of the light source 120′. In the following, the above difference will be mainly described and redundant descriptions of the same features will be omitted.

The light source 120′ may include a plurality of light sources. In an embodiment, the light source 120′ may include a first light source 121 and a second light source 122.

The first light source 121 and the second light source 122 may be separate optical devices, in which the first light source 121 may emit first input light L1 to the fluid receptacle 110, and the second light source 122 may emit second input light L2 to the fluid receptacle 110.

At least one of the first light source 121 or the second light source 122 may emit monochromatic or multicolor input light. For example, at least one of the first light source 121 or the second light source 122 may include a source device which generates monochromatic light, such as a gas laser, a semiconductor laser, or a laser diode, or at least one of the first light source 121 or the second light source 122 may be a device which produces multicolor light, such as a halogen lamp, a xenon lamp, or a white light-emitting diode.

In an embodiment, the first light source 121 and the second light source 122 may generate different wavelengths of input light. For example, the first light source 121 and the second light source 122 may include light filters through which different wavelengths of light may pass, respectively.

The positions and orientations of the first light source 121 and the second light source 122 may be adjusted independently. The biofilm measuring device 100′ according to another embodiment of the disclosure may adjust the intensity and the angle of incidence of the first input light L1 and the intensity and the angle of incidence of the second input light L2 emitted to the fluid receptacle 110, respectively, by adjusting the positions and orientations of the first light source 121 and the second light source 122 depending on the type of the target material P, the type of the input light L1 and L2, the size of the fluid receptacle 110, and the like, thereby causing speckles to be formed more actively in the fluid receptacle 110.

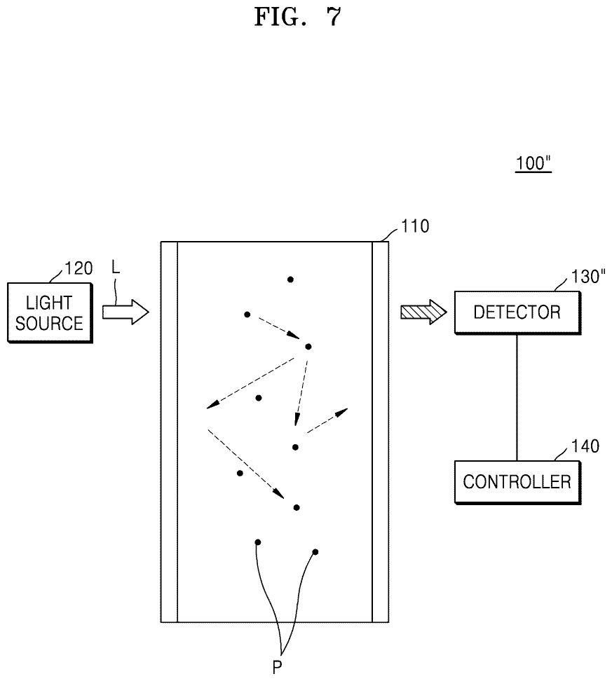

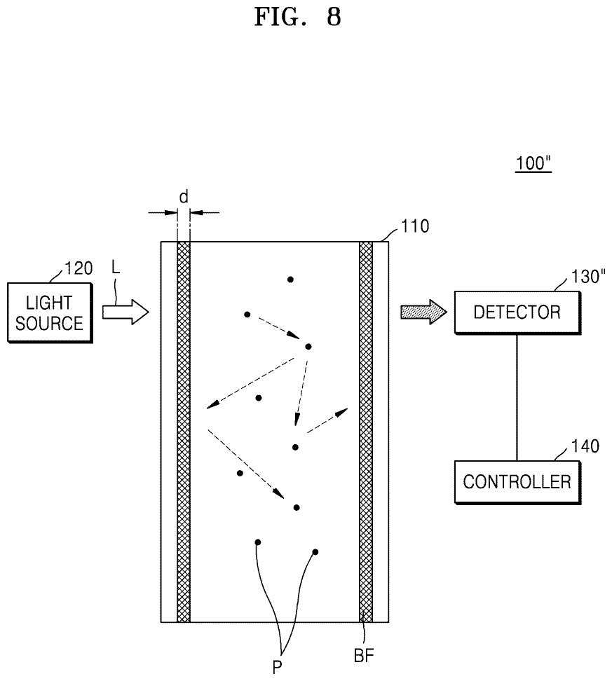

FIGS. 7 and 8 are conceptual views schematically illustrating a biofilm thickness measuring device 100″ according to another embodiment of the disclosure.

Referring to FIGS. 7 and 8, a biofilm thickness measuring device 100″ according to another embodiment of the disclosure may include a fluid receptacle 110, a light source 120, a detector 130″ and a controller 140. Compared to the biofilm thickness measuring device 100 according to an embodiment described with reference to FIGS. 1 to 3, the biofilm thickness measuring device 100″ according to another embodiment shown in FIGS. 7 and 8 differs in the configuration of the detector 130″. In the following, the above difference will be mainly described and redundant descriptions of the same features will be omitted.

The detector 130″ may be provided as a single detector. The detector 130″ may be provided as a device capable of simultaneously measuring speckles of the output light and the intensity of the output light. For example, the detector 130″ may be, but is not limited to, a CCD camera, and may be implemented as various types of image sensors capable of simultaneously measuring a speckle image and the intensity of the output light. The detector 130″ may obtain a plurality of images by measuring the output light in a time series sequence.

The controller 140 may calculate a temporal correlation of speckles of the output light using the plurality of images obtained by measuring the output light in a time series sequence, and calculate the concentration of the target material P contained in the target fluid using the calculated temporal correlation of speckles.

In an embodiment, the controller 140 may obtain intensity information of the output light by comparing an image measured at a first time point before the formation of the biofilm BF and an image measured at a second time point after the formation of the biofilm BF among the plurality of images obtained by measuring the output light in a time series sequence.

In another example, the controller 140 may obtain the intensity information of the output light using any of the plurality of images. In another example, the controller 140 may obtain the intensity information of the output light by calculating an average value using the plurality of images measured over a selected time period.

The controller 140 may calculate refractive index information of the biofilm BF using the calculated concentration of the target material P and the intensity information of the output light, and may calculate the thickness d of the biofilm BF using the calculated refractive index information of the biofilm BF.

Although the disclosure has been described with reference to the exemplary embodiments illustrated in the drawings, the embodiments are provided for illustrative purposes only, and a person of ordinary skill in the art will understand that various modifications and modified embodiments are possible therefrom. Accordingly, the true scope of the disclosure shall be defined only by the technical idea of the appended claims.

### Description of Reference Numbers

100: biofilm thickness measuring device

110: fluid receptacle

120: light source

130: detector

131: first detector

132: second detector

140: controller

What is claimed is:

1. A biofilm thickness measuring device comprising:
a fluid receptacle configured to contain a target fluid;
a light source configured to emit interfering input light toward the fluid receptacle;
a first detector configured to detect a speckle of output light produced by multiple scattering of the emitted input light in the target fluid;
a second detector configured to detect an intensity of the output light; and
a controller configured to calculate thickness information of a biofilm in the fluid receptacle using the detected speckle and the detected intensity of the output light.

2. The biofilm thickness measuring device of claim 1,
wherein the controller is further configured to obtain the intensity and the speckle of the output light in a time series sequence, and calculate refractive index information of the biofilm using a temporal change in the intensity of the output light and a temporal change in the speckle of the output light.

3. The biofilm thickness measuring device of claim 2,
wherein the controller is further configured to calculate the refractive index information by comparing the intensity of the output light measured at a first time point before formation of the biofilm on an inner surface of the fluid receptacle and the intensity of the output light measured at a second time point after the formation of the biofilm on the inner surface of the fluid receptacle.

4. The biofilm thickness measuring device of claim 2,
wherein the controller is further configured to calculate a thickness of the biofilm using calculated refractive index information of the biofilm.

5. The biofilm thickness measuring device of claim 1,
wherein the controller is further configured to obtain a temporal correlation of speckle using the detected speckle of the output light, and calculate a concentration of a target material in the target fluid based on the obtained temporal correlation.

6. The biofilm thickness measuring device of claim 5,
wherein the temporal correlation comprises a difference between first image information of the speckle detected at a first time point and second image information of the speckle detected at a second time point different from the first time point.

7. The biofilm thickness measuring device of claim 6,
wherein the first image information and the second image information comprise pattern information of the speckle.

8. The biofilm thickness measuring device of claim 1,
wherein the controller is further configured to obtain a spatial correlation of the speckle using the detected speckle of the output light, and calculate a concentration of a target material in the target fluid based on a temporal change of the obtained spatial correlation.

9. A biofilm thickness measuring device comprising:
a fluid receptacle configured to contain a target fluid;
a light source configured to emit interfering input light toward the fluid receptacle;
a detector configured to obtain an image of output light produced by multiple scattering of the emitted input light in the target fluid; and
a controller configured to obtain a speckle and an intensity of the output light using the obtained image of the output light, and calculate thickness information of a biofilm in the fluid receptacle using the obtained speckle and the obtained intensity of the output light.

10. The biofilm thickness measuring device of claim 9,
wherein the detector obtains a plurality of images by measuring the output light in a time series sequence, and
the controller is further configured to obtain an intensity of the output light by comparing an image measured at a first time point before formation of the biofilm on an inner surface of the fluid receptacle and an image measured at a second time point after the formation of the biofilm on the inner surface of the fluid receptacle among the plurality of images.
